(19) United States
(12) Patent Application Publication
(10) Pub. No.: US 2007/0128209 A1
(43) Pub. Date: Jun. 7, 2007

(54) VACCINE BASED METHOD FOR PROTECTION AGAINST HIV INFECTION

(75) Inventor: Geoffrey William Hoffmann, Vancouver (CA)

Correspondence Address: VERMETTE & CO., SUITE 320 - 1177 WEST HASTINGS STREET, VANCOUVER, BC, V6E2K3, CA

(73) Assignee: NETWORK IMMUNOLOGY INC., Vancouver (CA)

(21) Appl. No.: 11/309,193

(22) Filed: Jul. 12, 2006

Related U.S. Application Data
(63) Continuation-in-part of application No. 11/307,951, filed on Feb. 28, 2006.
(60) Provisional application No. 60/594,231, filed on Mar. 21, 2005.

(51) Int. Cl. A61K 39/00; A61K 39/21

(52) U.S. Cl. 424/185.1; 424/188.1

(57) ABSTRACT

A vaccine based method for the prevention of HIV infection, suitable for HIV negative persons, is described. The method comprises the combination of (a) an immunization with material containing foreign MHC class II and (b) receiving a preparation containing the same foreign MHC class II, for example (i) taken orally and optimally taken at the time of, or immediately before, or immediately after, exposure or possible exposure to HIV or (ii) applied to the vagina or the anus at, or close to, times of exposure and times of possible exposure to HIV.

### RELATED APPLICATIONS

This application is a continuation-in-part of previously filed U.S. patent application Ser. No. 11/307,951, filed on Feb. 28, 2006, which claimed the benefit of previously filed U.S. Provisional Patent Application No. 60/594,231, filed on Mar. 21, 2005 and U.S. patent application Ser. No. 11/307,951 filed Feb. 28, 2006.

### FIELD OF THE INVENTION

The present invention concerns a two component, vaccine based method for protecting people who are HIV negative from becoming infected with HIV. HIV is human immunodeficiency virus. HIV causes acquired immunodeficiency syndrome (AIDS) in people infected with the virus.

### BACKGROUND TO THE INVENTION

All publications mentioned hereunder are incorporated herein by reference.

A vaccine that prevents infection with HIV is urgently needed. Many approaches have been tried and failed. The classical approaches involve inducing immunity to HIV proteins.

Experiments have shown that xeoimmunity and alloimmunity can be protective against HIV infection and SIV infection. Stott et al. 1991, Nature 353, 393; Arthur et al. 1995 J. Virol. 69, 3117-3124; MacDonald et al. 1998, J. Infect. Dis. 177, 551-556; Wang et al. 1999, Nature Medicine, 5, 1004-1009; Spear et al. 2001, J. Acquir. Immune Defic. Syndr. 26, 103-110; Leith et al. 2003, Aids Res. Hum. Retrovir. 19, 957-965. Peters et al. 2004, Lancet, 363, 518-524. SIV is simian immunodeficiency virus, a virus that is similar to HIV, and causes an AIDS-like disease in macaque monkeys. In the experiment of Arthur et al. it was shown that sterilizing immunity against SIV infection can result from immunity to human MHC class II. The protection was against challenge with SIV grown in human cells. However these macaques were not protected against challenge with SIV grown in macaque cells.

The symmetrical network theory of immune system regulation includes a theory of AIDS pathogenesis. Papers on the symmetrical network theory include Hoffmann 1975, Eur. J. Immunol. 5, 638-647; Hoffmann 1978, in “Theoretical Immunology”, Bell et al. eds., Marcel Dekker, N.Y., 571-602; Hoffmann 1979, in Lecture Notes in Biomathematics Springer-Verlag vol. 32, Bruni et al. eds. 239-257; Hoffmann 1981, in “The Immune System, Festschrift in Honour of Niels Kaj Jerne, on the Occasion of his 70th Birthday”, Lefkovits et al. eds. Karger, Basel, 1981, vol. I, 28-34; Gunther et al. 1982, Journal of Theoretical Biology, 94, 815-855; Hoffmann 1982, in Regulation of Immune Response Dynamics, DeLisi et al. eds, CRC Press, 137-162; Cooper-Willis et al. 1983, Mol. Immunol. 20, 865-870; Hoffmann et al. 1986, J. Immunol. 137, 61-68; Hoffmann 1988, in “The Semiotics of Cellular Communication in the Immune System”, Sercarz et al. eds. Springer-Verlag, New York 257-271; Hoffmann et al. 1988, in “Theoretical Immunology, Part Two”, Perelson, ed., Santa Fe Institute Series “Studies in the Science of Complexity”, Addison Wesley Publishing Company, 291-319; Grant et al. 1989, in “Cellular Basis of Immune Modulation”, Kaplan et al. eds., A. R. Liss, Inc., New York, 481-484; Hoffmann et al. 1988, Canadian Research 21, No. 6, 16-23; Grant, M. D. et al. 1990, J. Immunol., 144, 1241-1250; Forsyth et al. 1990, J. Immunol., 145, 215-223; Hoffmann et al. 1990, in “Idiotype Networks in Biology and Medicine”, Osterhaus et al. eds, Elsevier, Amsterdam 295-299; Hoffmann et al. 1991, Proc. Nat. Acad. Sci. (USA), 88, 3060-3064; Kion et al. 1991, Science 253, 1138-1140; Hoffmann et al. 1993, in “New Concepts in AIDS Pathogenesis” L. Montagnier et al. eds. Marcel Dekker New York, 273-290; Hoffmann 1994, Immunology and Cell Biology, 72, 338-346.

### SUMMARY OF THE INVENTION

The present invention comprises a vaccine for the prevention of HIV infection in humans, wherein the vaccine contains foreign MHC class II molecules. The foreign MHC class II can be xenogeneic MHC class II, allogeneic MHC class II or MHC class II that has been chemically modified. The foreign MHC class II molecules may be in the form of aggregates or multimers (e.g. tetramers), they may be non-human MHC class II molecules, (e.g. from a species that is more phylogenetically distant from humans than humans are phylogenetically distant from macaque monkeys, such as a mouse) and/or they may be chemically modified. The vaccine may additionally contain an adjuvant.

The present invention additionally contemplates a method of using the vaccine for preventing HIV infection in an individual. The method involves administering to an individual a vaccine containing foreign MHC class II molecules, and administering to the individual a further dose of the foreign MHC class II molecules close in time to a possible HIV exposure event. The additional dose may be administered in a number of ways, including orally or applied to the vagina or anus.

### BRIEF DESCRIPTION OF THE DRAWING

Further features and advantages will be apparent from the following Detailed Description of the Invention, given by way of example, of a preferred embodiment taken in conjunction with the accompanying drawing, wherein:

FIG. 1: This FIGURE shows a model of HIV pathogenesis, which underlies the invention. Helper T cells are selected to have some complementarity to MHC class II. Suppressor T cells are selected such that their V regions have complementarity to as many helper T cell V regions as possible. In addition to CD4, CXCR4 and CCR5, the T cell receptor of HIV-specific helper T cells is a coreceptor for HIV. HIV variants that are recognized by the largest number of helper T cells are preferentially produced, and also stimulate these helper T cells to proliferate. In other words, there is co-selection of helper T cells and HIV variants. There is also co-selection of helper T cells and suppressor T cells. Since HIV and the suppressor T cells are subject to the same selection criterion, there is a natural selection process in which HIV proteins and the V regions of the suppressor T cells converge in shape space. Anti-HIV immunity then becomes specific for the suppressor T cell quasi-species. Immunity against this central regulating element of the system results in the collapse of the system. The suppressor T cells no longer adequately stimulate and regulate the helper T cells, that are then free to inappropriately help immune responses against self components, and autoimmunity ensues. The autoimmunity can include immunity directed against helper T cells that express CD4.

### DETAILED DESCRIPTION OF THE INVENTION

Much evidence supports the concept that AIDS is an autoimmune disease triggered by HIV. Kion et al. 1991, Science, 253, 1138-1140; Bourinbaiar et al. 2005, Autoimmunity Reviews 4, 403-409. A model of AIDS pathogenesis explains how the interaction of HIV with the immune system results in the collapse of a central regulating component of the T cell repertoire, leading to autoimmunity. Hoffmann 1994, Immunol. and Cell Biol., 72, 338-346; Hoffmann, 1995, Scand. J. Immunol., 41, 331-337.

The experiments that have shown that xenoimmunity and alloimmunity can be protective against HIV infection and SIV infection lead to a new interpretation of the model of Hoffmann, 1994, op. cit, which is the basis of the present invention. A review of that model follows.

A “quasi-species” is a diverse population of similar elements. The term was originally coined to refer to related macromolecules in an origin of life scenario. Eigen et al. 1988, J. Phys. Chem. 92, 6881-6891. HIV is a rapidly mutating virus, and a consequently diverse array of HIV virions can be referred to as a quasi-species.

“Co-selection” refers to mutual selection of members within two diverse populations, as the result of some sort of complementarity in shapes of members of one of the populations to shapes in the other population. Hoffmann 1994, Immunol. and Cell Biol., 72, 338-346.

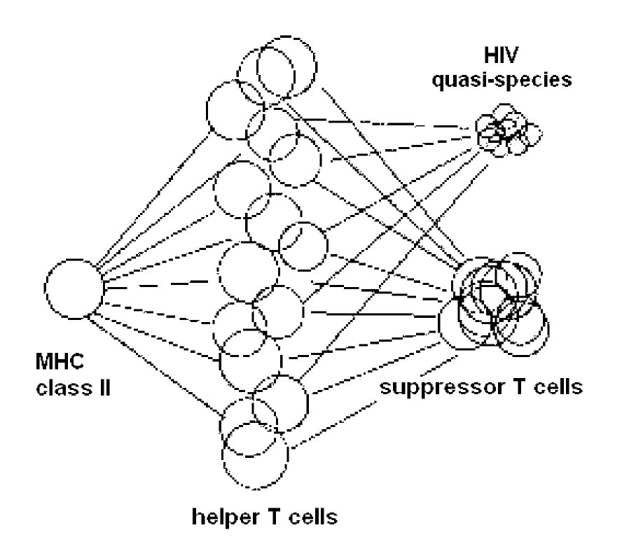

Helper T cells are highly diverse, but are nevertheless generally selected to have some affinity for MHC class II. In the model shown in FIG. 1 there are suppressor T cells that are selected according to the criterion of being able to recognise and be idiotypically stimulated by as many helper T cells as possible. The suppressor T cell population is then a quasi-species, with the similarity in its idiotypes being the result of the uniform selection criterion for all of these cells. There is co-selection of helper T cells and the suppressor T cells.

A key postulate of the model is that the antigen-specific helper T cell receptor is a coreceptor for infection by HIV. Hoffmann, op. cit 1994. This means that helper T cells that specifically recognise the virus are preferentially infected, and these helper T cells are also stimulated by HIV to proliferate. This postulate has been validated. Douek et al. 2002 Nature 417, 95-98. Strains of HIV that are recognised by the largest number of helper T cells preferentially replicate. We then have co-selection also of HIV and helper T cells.

HIV and the suppressor T cell population are then subject to the same selection pressure, namely to recognise as many helper T cell idiotypes as possible. The consequence in many cases is that the HIV quasi-species and the suppressor cell quasi-species converge in shape space, meaning that the average shape of the HIV population looks more and more like the average shape of the suppressor T cell idiotypes. At the same time there is anti-HIV immunity. This immunity then cross-reacts with the idiotypes of the suppressor T cell population, and this centrally regulating element of the system comes under attack from the anti-HIV immunity. The helper cells are then no longer adequately regulated, and autoimmunity ensues. The model is directly supported by some remarkable findings that link autoimmunity, alloimmunity, idiotypic network regulation and AIDS. Kion et al. 1991, op cit. Autoimmunity in AIDS can include cytotoxic T cell activity against CD4 helper T cells. Grant et al. 1996, Immunol. and Cell Biol. 74, 38-44.

The conclusion that was previously derived from this model was that we should endeavour to eliminate HIV-specific T cells. Hoffmann, 1995, op. cit. That may be extremely difficult, given the diversity of the virus and the diversity of the helper T cells. Surprisingly, a new interpretation leads to a very different vaccine based method for the prevention of HIV infection, which is the subject of the present invention.

The method of the present invention involves one or more vaccinations with foreign MHC class II to induce a specific IgG response, followed by one or more doses of the same foreign MHC class II at, or close to, times of exposure and possible exposure to HIV. The foreign MHC class II can be xenogeneic MHC class II, allogeneic MHC class II or MHC class II that has been chemically modified. The doses of foreign MHC class II taken at, or close to, the times of exposure and times of possible exposure to HIV material may, for example, be taken orally, applied to the vagina, or applied to the anus.

Chemically modified MHC class II is MHC class II that has been chemically modified, such that it is capable of inducing an immune response in an animal or person that expresses MHC class II with the same amino acid sequence. One form of such modification is that a peptide is covalently or non-covalently bound in the cleft between the α and β chains of the MHC class II molecule. The foreign MHC class II in the dose that is taken orally or applied to the vagina or anus at times of exposure and times of possible exposure to HIV is the same or substantially the same MHC class II as used in the vaccination, in the sense that it has the same or substantially the same amino acid sequence, and has the same or substantially the same modification if it is chemically modified MHC class II. Substantially the same here means that the foreign MHC class II used in the two parts of the method have sufficient identical immunogenic portions such that they stimulate substantially the same set of helper T cells. The initial vaccination or vaccinations with foreign MHC class II to induce a specific IgG response can include an adjuvant. In the preferred embodiment of the method, the foreign MHC class II molecules in the dose(s) are identical to the foreign MHC class II in the vaccination(s).

Helper T cells that recognise both HIV and suppressor cell idiotypes play a key role in the convergent selection process described above. When HIV infection occurs, those clones are especially selected, and they lead to the dominant selection of HIV variants and suppressor cell clones that resemble each other. The average shape of the HIV quasi-species thus undergoes selection in virus carriers to resemble the average shape of the suppressor T cell idiotypes. If however the average shape of the suppressor T cell idiotypes in an individual differs sufficiently from the average shape of the HIV with which an individual is infected, the population of helper T cells with which HIV interacts may be effectively distinct from the population interacting with the suppressor T cells. Then there are two separate co-selection processes, that do not necessarily converge.

Live xenogeneic cells and allogeneic cells are potent antigens. Hence xenoimmunization and alloimmunization cause substantial changes to the T cell and B cell repertoires. The average shape of the specific receptors of the helper T cell population changes, or moves in shape space, with the consequence that the average shape of the specific receptors of the co-selected suppressor cell population moves in shape space. This shift can be sufficient, such that the infecting HIV then interacts with a population of helper T cells distinct from the population with which the suppressor T cells interact. The convergence of HIV and the suppressor cell population then does not occur.

In an experiment by Arthur et al. two macaques were immunized four times with human MHC class II, and two weeks after the final immunization they were challenged with SIV that had been propagated in human cells. Both animals were fully protected against this challenge, including being negative by recovery of virus and by PCR. Hence immunity to xenogeneic MHC class II was protective in this case (sterilizing immunity). The macaques were subsequently boosted by another immunization with human MHC class II, and two weeks later were challenged with SIV that had been propagated in macaque cells. They both became infected. In the context of the theory underlying the present invention, the failure of the vaccine in this case is attributed to SIV and MHC molecules in the challenge preparation stimulating a different set of helper T cells, rather than the set that was selected by the vaccine. The new set of helpers select a different set of suppressors. There is a positive feedback loop for this second set of helper and suppressor T cells, and this second positive feedback loop competes against the positive feedback loop of the vaccine-specific helper T cells and their corresponding co-selected suppressor T cells. The present invention provides a method for overcoming the competition of the second positive feedback loop by boosting the first feedback loop with a dose of the foreign MHC class II of the vaccine given at an appropriate time point.

The theory underlying the invention then predicts firstly that if a macaque is immunized with foreign MHC class II, such that it makes an IgG response to the foreign MHC class II, and the macaque is challenged with SIV that has been passaged in macaque cells, and at the time of this challenge the macaque receives a dose of the same or substantially the same foreign MHC class II, the macaque is protected against infection with SIV. Similarly, the present invention prescribes that a human who is immunized with foreign MHC class II, such that the human makes an IgG response to the foreign MHC class II, and the human is exposed to infection with HIV, and at a time point close to the time of being exposed to HIV the human receives a dose of the same or substantially the same foreign MHC class II, for example taken orally, or applied to the vagina or the anus, the human is protected against infection with HIV.

This method works because the dose of the foreign MHC class II given at or near the time of exposure to HIV stimulates the vaccine-specific helper T cells, and hence indirectly their corresponding suppressor T cells. These vaccine-specific helper T cells and the corresponding suppressor T cell population then dominate the competition mentioned above. The suppressor T cell population is then again distinctly different in shape from the potentially infecting HIV, and sterilizing immunity directed against HIV again is not also immunity directed also against this central regulating element.

As stated, in the case of a macaque experiment the additional dose with the vaccine MHC class II material can be given at the same time as the challenge with virus. In the case of preventing HIV infection in humans, preferred embodiments of the invention are that additional doses of foreign MHC class II, given at times of exposure and times of possible exposure to HIV, are given in oral form or are applied to the vagina or the anus, optimally at the time of or immediately prior to or immediately following possible exposure to HIV. How close to the time of possible exposure to HIV the boost must be given for efficacy can be more definitively determined by experiment. Within twelve hours of the possible exposure, for example, may suffice.

The competition between the two positive feedback loops is envisaged as occurring at the surface of accessory cells (“A cells”) such as macrophages. T cells secrete antigen-specific and antiidiotypic T cell factors that are cytophilic for A cell surfaces. Evans et al. 1972, J. Exp. Med., 136, 1318-1322. Mutual stimulation of antigen-specific and corresponding antiidiotypic T cells catalysed by A cells is part of the symmetrical network theory of the regulation of the immune system. Hoffmann 1978, op. cit. When the immune system has been immunized with a foreign MHC class II vaccine, and it is boosted with the same foreign MHC class II, T cell factors that are specific for the foreign MHC class II bind to the surface of the A cell and constitute an immunogenic array for the corresponding antiidiotypic suppressor T cells. The stimulation of these suppressor T cells leads to the secretion of antiidiotypic T cell factors, that also bind to the surface of A cells and further stimulate the foreign MHC class II vaccine-specific helper T cells, completing a positive feedback loop. That is, the A cells function as catalysts for the mutual stimulation and hence selection of the foreign MHC class II vaccine-specific helper T cells and the corresponding antiidiotypic suppressor T cells. If, at close to the same time, the immune system is stimulated by a second type of foreign MHC antigens, (i.e. different from the foreign MHC class II in the vaccine) for which the system has not been immunized, the surfaces of A cells cannot simultaneously act as catalysts for those second type antigens, since their second type antigen-specific T cell factors are greatly outnumbered by the first foreign MHC class II vaccine-specific and corresponding antiidiotypic T cell factors. An indication of the rapidity of the A cell catalysed mutual stimulation of antigen-specific and antiidiotypic T cells, leading towards an unresponsive or suppressed state for the T cells, ascribed in the theory to the system being on a trajectory towards elevated levels of antigen-specific and corresponding antiidiotypic T cells, is given by an experiment involving the kinetics of induction of unresposiveness in T cells and B cells. Chiller et al. 1971, Science 171, 813-815. The T cells were essentially unresponsive to the antigen in the context of an adoptive transfer experiment within about a day.

The vaccine of the present invention does not need to contain live cells or dead cells. The main component in the vaccine, that causes the required shift in the T cell repertoires, is foreign MHC class II molecules. The foreign MHC class II can be xenogeneic MHC class II or a chemically modified version of human or xenogeneic MHC class II. Allogeneic MHC class II molecules that are present in some members of the population are not a preferred embodiment of the invention, because for a given human MHC class II molecule, some individuals will normally have that molecule as a self antigen, and hence immunization of those individuals with it will not cause the required shift.

In a preferred embodiment the vaccine of the present invention contains xenogeneic MHC class II molecules from a species that is more phylogenetically different from both humans and macaque monkeys, than humans are phylogenetically different from macaque monkeys. For example, the vaccine can consist of mouse MHC class II. The immune response of a macaque to mouse MHC class II is similar to that of a human to mouse MHC class II. This vaccine based method, using for example mouse MHC class II, is expected to prevent both SIV infection in macaques and HIV infection in humans. The same agent or agents can therefore go directly from an animal trial in macaques to clinical trials in humans. This enables optimally rapid development of this vaccine based method.

An advantage of using mouse MHC class II is that the immune response of a human to mouse MHC class II is similar to the immune response of a mouse to human MHC class II, since the phylogenetic distance is exactly the same in both cases. Parameters of immune responses in mice to immunizations with human MHC class II are therefore predictive of parameters for converse immunizations of humans with mouse MHC class II. This advantage is not shared by chemically modified MHC class II. Furthermore, the mouse and human immune systems are the two systems that have been studied most extensively by immunologists.

The MHC class II vaccine may be given with or without an adjuvant. The possible need for an adjuvant can be assessed, by one skilled in the art, for example in experiments in which mice are immunized with human MHC class II, and tested for the production of IgG antibodies specific for the human MHC class II.

MHC class II molecules are heterodimers, each consisting of an α and a β chain. In one embodiment of the invention multimers of these heterodimers are used. Multimers are potentially more effective in cross-linking T cell receptors and are therefore potentially more immunogenic than the simple MHC class II heterodimers. For example, MHC class II tetramers are available, that have been produced for other purposes.

In one embodiment of the invention the MHC class II molecules are aggregated to make them more immunogenic. The need for such aggregation can be assessed by one skilled in the art, for example in experiments in which mice are immunized with human MHC class II.

As stated, the invention requires the use of MHC class II molecules that are different from the MHC class II of the immunized person, that is, foreign MHC class II. Foreign MHC class II includes xenogeneic MHC class II, allogeneic MHC class II, and chemically modified versions of the immunized person's MHC class II. There is a practically unlimited number of ways in which MHC class II can be chemically modified.

The vaccine based method of the present invention is strictly for people who are not already infected with HIV. It is not effective for people who are HIV positive. Furthermore, giving the vaccine to HIV positive people may result in the emergence of HIV strains against which the present vaccine based method is ineffective. The vaccine is therefore to be given to individuals only after they have been given an HIV test, and have been shown to be HIV negative.

What is claimed is:

1. A vaccine for the prevention of HIV infection in humans, wherein said vaccine contains foreign MHC class II molecules.

2. The vaccine of claim 1, wherein said foreign MHC class II molecules are multimers.

3. The vaccine of claim 1 or 2, wherein said foreign MHC class II molecules are tetramers.

4. The vaccine of one of claims 1 to 3, wherein said foreign MHC class II molecules are non-human MHC class II molecules.

5. The vaccine of one of claims 1 to 4, wherein said foreign MHC class II molecules are from a species that is more phylogenetically distant from humans than humans are phylogenetically distant from macaque monkeys.

6. The vaccine of one of claims 1 to 5, wherein said foreign MHC class II molecules are mouse MHC class II molecules.

7. The vaccine of one of claims 1 to 6, wherein said foreign MHC class II molecules are in aggregated form.

8. The vaccine of one of claims 1 to 7, wherein the vaccine additionally contains an adjuvant.

9. The vaccine of one of claims 1 to 8, wherein said foreign MHC class II molecules are chemically modified.

10. A method for preventing HIV infection in an individual, comprising:
(a) administering to the individual a vaccine containing foreign MHC class II molecules, wherein said foreign MHC class II molecules are one of xenogeneic MHC class II molecules, allogeneic MHC molecules and chemically modified MHC class II molecules, such that the individual makes a specific IgG response to the MHC class II molecules; and
(b) administering to the individual a dose of foreign MHC class II molecules, close in time to a possible HIV exposure event, wherein said exposure event exposes said individual to risk of HIV infection, and wherein said foreign MHC class II molecules in said dose are the same or substantially the same as said foreign MHC class II molecules in said vaccine.

11. The method of one of claim 10, wherein the vaccine contains an adjuvant.

12. The method of claim 11 or 10 wherein said dose is taken orally or applied to the vagina or applied to the anus.

13. The method of one of claims 10 to 12, wherein said foreign MHC class II molecules are multimers.

14. The method of one of claims 10 to 13, wherein said foreign MHC class II molecules are tetramers.

15. The method of one of claims 10 to 14, wherein said foreign MHC class II molecules are non-human MHC class II molecules.

16. The method of one of claims 10 to 15, wherein said foreign MHC class II molecules are from a species that is more phylogenetically distant from humans than humans are phylogenetically distant from macaque monkeys.

17. The method of one of claims 10 to 16, wherein said foreign MHC class II molecules are mouse MHC class II molecules.

18. The method of one of claims 10 to 17, wherein said foreign MHC class II molecules are in aggregated form.

19. The method of one of claims 10 to 18, wherein said vaccine is administered to said individual more than once.
